(19) United States
(12) Patent Application Publication
(10) Pub. No.: US 2009/0142796 A1
(43) Pub. Date: Jun. 4, 2009

(54) Detection of Inducible Resistance to Macrolide-Lincosamide-Streptogramin b

(75) Inventors: Charles Yu, Lutherville, MD (US); Curtis Michael Gosnell, Fallston, MD (US); Vicki Whitley, New Freedom, PA (US); David J. Turner, Owings Mills, MD (US)

Correspondence Address: David W. Highet, VP & Chief IP Counsel;Becton, Dickinson and Company, 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/950,388

(22) Filed: Dec. 4, 2007

(51) Int. Cl. C12Q 1/18; C12M 1/22

(52) U.S. Cl. 435/32; 435/288.4

(57) ABSTRACT

The embodiments of this invention provide a test panel and method for the detection of inducible resistance to macrolide-lincosamide-streptogramin b (iMLSb) in microorganisms in an automated microorganism identification (ID) and antimicrobial susceptibility determinations (AST) system.

The wells of a multiple-well test panel contain a macrolide agent and a lincosamide agent and a combination of both macrolide agent and lincosamide agent. The test panel is inoculated with a broth-suspended microorganism, and placed into the automated microorganism identification (ID) and antimicrobial susceptibility determinations (AST) system. The test panel is incubated within the system and the wells monitored for microorganism growth.

### BACKGROUND OF THE INVENTION

Many conventional systems exist for performing tests on microbiological samples related to patient diagnosis and therapy. The microorganism samples may come from a variety of sources, including infected wounds, genital infections, cerebro-spinal fluids, blood and abscesses. From those microorganism samples an inoculum is prepared in accordance with established procedures which produce a bacterial or cellular suspension of a predetermined concentration. Further processing of the suspension may depend on the testing method employed.

These systems are used, for example, for identification of which microorganisms are present in a patient's sample. Typically, in such systems, reagents are placed into cupules, or test wells, of identification trays, which in the presence of an actively growing culture of microorganisms change color. Based on the color change, or lack thereof, the microorganism can be identified by the use of reference tables.

Other systems have been developed for susceptibility testing of microorganisms. These systems are used to determine the susceptibility of a microorganism in a sample to various therapeutics, such as antibiotics. Based on these test results, physicians can then, for example, prescribe an antimicrobial product which will be successful in killing or inhibiting the microorganism. In particular, qualitative susceptibility testing produces an indication of whether a microorganism is resistant or sensitive to a particular antibiotic, but does not provide an indication on the degree of sensitivity or resistance of the microorganism. On the other hand, quantitative susceptibility testing, provides an indication of the concentration of the antimicrobial agent needed to inhibit growth of the microorganism. The term minimum inhibitory concentration (MIC) is used to refer to the minimum concentration of the antimicrobial agent that is required to inhibit the growth of the microorganism.

Automated systems are desirable in performing these tests to minimize the technician handling time, as well as to minimize the possibility of human error. In addition, automated systems that obtain results rapidly and accurately are preferred.

U.S. Pat. No. 6,096,272; U.S. Pat. No. 6,372,485; and U.S. Pat. No. 7,115,384 the disclosures of which are hereby incorporated by reference, describe one such automated microbiological testing system that tests a plurality of multiple-well test panels, for identification and susceptibility, with a minimal amount of human intervention during the testing process. In addition, this system performs both colorimetric- and fluorometric-type testing. Moreover, this system quickly analyzes the gathered test data to produce accurate identification and/or susceptibility testing results.

Certain strains of staphyloccoci and/or streptococci may express an antibiotic resistance phenotype known as inducible-macrolide-lincosamide-streptogramin b (iMLSb). These microorganisms were shown to possess an erm gene, and will express the gene product only when the microorganism is grown under an inducing condition. The Clinical and Laboratory Standard Institute (CLSI) recommended the use of “D-test” for detection of iMLSb phenotype. “D-test” is based on the knowledge that the presence of erythromycin in the growth medium will induce the expression of the erm gene.

However in order for “D-test” to be diagnostically valuable for detection of iMLSb phenotype, the microorganism must first be tested to confirm resistance to erythromycin and susceptibility to clindamycin.

To perform a “D-test” one has to first make a standardized suspension of the test microorganism. Then a thin lawn of the microorganism is plated onto an agar plate. A filter disk impregnated with a specific amount of erythromycin is placed onto the lawn. A second filter disk impregnated with a specific amount of clindamycin will be placed onto the same lawn at a fixed distance from the erythromycin disk. The agar plate is then incubated overnight. During the incubation, antibiotics in the filter disks will diffuse into the agar medium, forming a gradient of antibiotic across the agar plate. Due to the proximity of the erythromycin disk and the clindamycin disk to each other, a composite gradient of the two antibiotics is formed in the agar between the two disks. If the test microorganism possesses an inducible erm gene, it will become more resistance to clindamycin in the area between the two disks, thus forming a “D” shape growth inhibition zone. The D-test is a highly effective method for detection of iMLSb phenotype but requires significant amount of manipulation of the test microorganism and antibiotic reagents and subjective interpretation of the test result.

A need exists for a user-friendly test for the detection of inducible resistance to macrolide-lincosamide-streptogramin b (iMLSb) in microorganisms in an automated antibiotic susceptibility testing (AST) system, eliminating the requirement of handling antibiotic disks.

### SUMMARY OF THE INVENTION

In one embodiment a test panel is provided for a testing system such as an automated microorganism identification (ID) and antimicrobial susceptibility determinations (AST) system, having a plurality of wells, a lincosamide agent, and a macrolide agent, wherein one or more of the wells contains both the lincosamide agent and the macrolide agent. An example of a suitable lincosamide agent is clindamycin and an example of a suitable macrolide agent is erythromycin.

In another embodiment a test panel is provided for a testing system such as an automated microorganism identification (ID) and antimicrobial susceptibility determinations (AST) system, having a plurality of wells, a lincosamide agent, and a macrolide agent, wherein one or more of the wells contains the lincosamide agent and is free of a macrolide agent and one or more of the wells contains the macrolide agent and is free of a lincosamide agent and one or more of the wells contains both the lincosamide agent and the macrolide agent.

In an additional embodiment a test panel is provided for a testing system such as an automated microorganism identification (ID) and antimicrobial susceptibility determinations (AST) system, having a plurality of wells, at least one lincosamide agent, and at least one macrolide agent, wherein one or more of the wells contains a lincosamide agent and is free of a macrolide agent and one or more of the wells contains a macrolide agent and is free of a lincosamide agent and one or more of the wells contains both a lincosamide agent and a macrolide agent.

In another embodiment a method is provided for the detection of inducible resistance to macrolide-lincosamide-streptogramin b (iMLSb) in microorganisms in a testing system by inoculating a test panel having a plurality of wells with a test microorganism, wherein one or more of the wells contains both a lincosamide agent and a macrolide agent and one or more of the wells contains a lincosamide agent and is free of a macrolide agent, and one or more of the wells contains a macrolide agent and is free of a lincosamide agent, placing the test panel into a testing system, incubating the test panel at an appropriate temperature, and monitoring the growth rate of the test microorganism in the wells, then comparing the growth rate of the test microorganism in the wells containing a lincosamide agent and free of a macrolide agent, and the growth rate of the test microorganism in the wells containing a macrolide agent and free of a lincosamide agent, and the growth rate of the test microorganism in the wells containing both a lincosamide agent and a macrolide agent. The presence of resistance to macrolide-lincosamide-streptogramin b (iMLSb) in the test microorganism is indicated by resistance in the wells containing a macrolide agent and free of a lincosamide agent, susceptibility in the wells containing a lincosamide agent and free of a macrolide agent, and resistance in said wells containing both a lincosamide agent and a macrolide agent.

In a further embodiment a method is provided for the detection of inducible resistance to macrolide-lincosamide-streptogramin b (iMLSb) in microorganisms known to be resistant to erythromycin and susceptible to clindamycin in an automated testing system by inoculating a test panel having a plurality of wells with a test microorganism, wherein one or more of the wells contains both a lincosamide agent and a macrolide agent, placing the test panel in the automated testing system, incubating the test panel at an appropriate temperature, and monitoring the growth rate of the test microorganism in the wells containing both the lincosamide agent and the macrolide agent. The presence of microorganism growth within one or more of the wells containing both the lincosamide agent and the macrolide agent, indicates the presence of resistance to macrolide-lincosamide-streptogramin b (iMLSb).

### BRIEF DESCRIPTION OF THE DRAWINGS

FIG. 1 is a front perspective view of a prior art system for performing microorganism identification (ID) and antimicrobial susceptibility determinations (AST) as disclosed in U.S. Pat. No. 6,096,272 with the enclosure door closed.

FIG. 2 is a front perspective view of a prior art system for performing microorganism identification (ID) and antimicrobial susceptibility determinations (AST) as disclosed in U.S. Pat. No. 6,096,272 with the enclosure door open.

FIG. 3A is a perspective view of an ID/AST test panel a prior art system for performing microorganism identification (ID) and antimicrobial susceptibility determinations (AST) as disclosed in U.S. Pat. No. 6,096,272.

FIG. 3B is a top view of an ID/AST test panel of a prior art system for performing microorganism identification (ID) and antimicrobial susceptibility determinations (AST) as disclosed in U.S. Pat. No. 6,096,272.

FIG. 3C is a bottom view of an ID/AST test panel of a prior art system for performing microorganism identification (ID) and antimicrobial susceptibility determinations (AST) as disclosed in U.S. Pat. No. 6,096,272.

FIG. 4 is a schematic top view of the internal components of the apparatus of FIG. 1 as disclosed in U.S. Pat. No. 6,096,272.

### DESCRIPTION

The embodiments of this invention provide a test panel and method for the detection of inducible resistance to macrolide-lincosamide-streptogramin b (iMLSb) in microorganisms in a testing system. Suitable testing systems include a microorganism identification (ID) testing system or an antimicrobial susceptibility determinations (AST) testing system or a combined microorganism identification (ID) and antimicrobial susceptibility determinations (AST) testing system. In addition the testing system may be an automatic, semi automatic or a manual testing system.

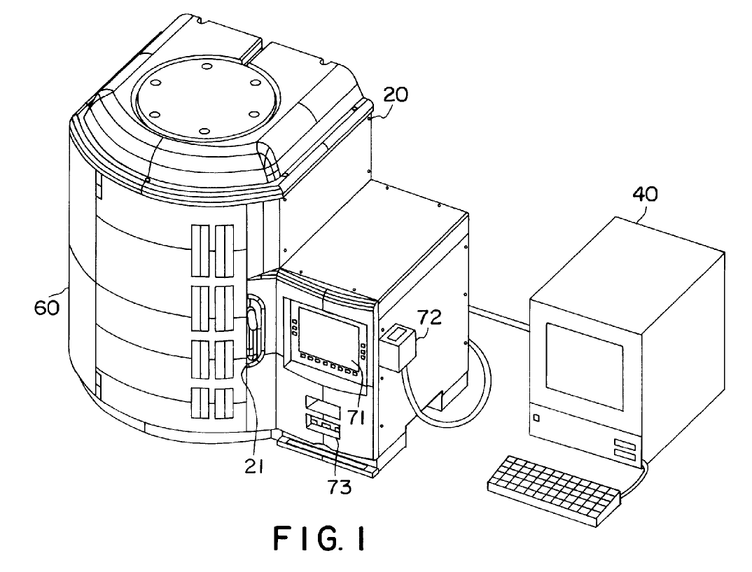

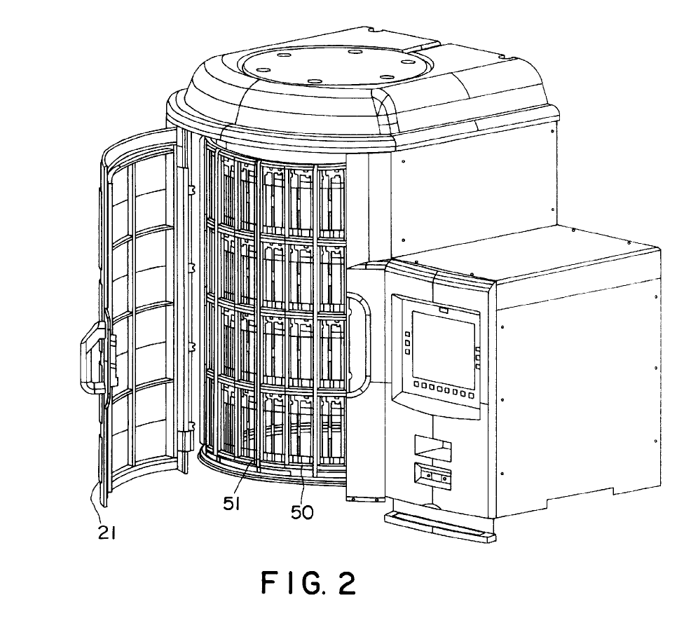

An example of a suitable automated testing system for performing highly reliable microorganism identification (ID) and antimicrobial susceptibility determinations (AST) is disclosed in U.S. Pat. No. 6,096,272 the disclosure of which is hereby incorporated by reference. This system determines identification and susceptibility based on readings from wells 31 contained in ID/AST panels 30 (see FIGS. 1 and 2). For example, in one embodiment, the wells 31 contain different reagent substrates and/or different antimicrobic dilutions which may change optical character sometime after being inoculated with the microorganism. The detection method described in U.S. Pat. No. 6,096,272 measures changes in absorption, scattering, and/or fluorescence. It may also measure luminescence. These changes are processed to determine the identification and susceptibility of the microorganism.

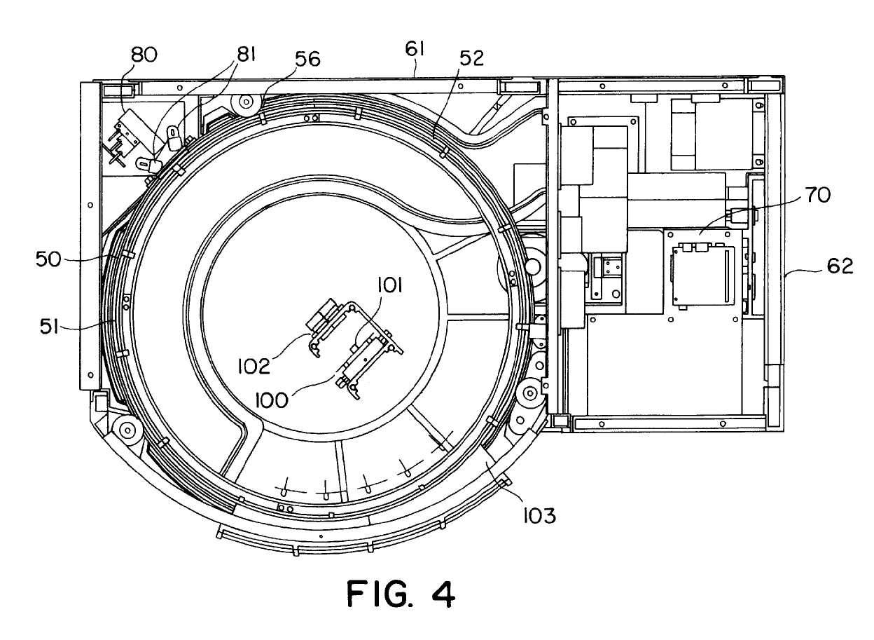

The system allows a technician, for example, after having inoculated the wells 31 of the ID/AST panel 30 with an unknown microorganism, to place that panel into an instrument 20 (shown in FIG. 4) where it is incubated at a set temperature, periodically interrogated for changes and analyzed for microorganism identification and antimicrobic susceptibility. The apparatus 20 holds a plurality of ID/AST panels 30 and provides positivity analysis results to the technician, as described below.

As shown in FIGS. 1-3, the ID/AST panels 30 are disposable devices which are inoculated with reagents needed for both ID and AST testing. The testing is performed on reactions generated by the samples and reagents placed in individual wells 31 on each ID/AST panel 30. The wells 31 are arranged on the ID/AST panels 30 as a two-dimensional array having rows and columns.

The instrument 20 is self-contained and sufficiently autonomous to test the ID/AST panels 30 and supply the appropriate test results. The instrument 20 stores, incubates and reads the ID/AST panels 30. The instrument 20 has a door 21 shown closed in FIG. 1 and open in FIG. 2 to allow for access to the interior of the instrument 20.

FIG. 1 shows, a personal computer (PC) workstation 40 is communicatively connected to the instrument 20. The PC workstation complements the instrument's 20 microbiology information system reporting and data management features, which are discussed below. The PC workstation 40 provides tools to improve empiric therapy decision and identify therapy intervention instances. The PC workstation 40 also incorporates reporting tools to assist infection control and epidemiology.

The instrument 20 includes a carousel 50, as shown in FIG. 2. The carousel 50 includes an assembly 51 comprised of rings and ribs bolted to a drive ring 52 to form a cylindrical cage. The carousel 50 is mounted vertically in an instrument enclosure 60 (shown in FIG. 1). The instrument enclosure 60 defines the carousel compartment 61 and an electronics compartment 62 (shown in FIG. 4). The carousel compartment 61 is insulated to provide a substantially uniform temperature incubation environment, and is light-tight under normal operation to prevent ambient light from entering.

Preferably, the carousel compartment 61 is continuously maintained at a temperature of 35° C. with the first and second predetermined set points being set at 39° C. and 33° C., respectively. However, as will be appreciated by one skilled in the art, other temperature settings may be used to achieve the particular testing requirements.

As shown in FIG. 4, a plurality of light source assemblies are mounted within the carousel compartment 61 and exterior to the circumference of the assembly 51. In a preferred embodiment of the present invention, the light source assemblies comprise a visible light source assembly 80 and an Ultra-Violet (UV) light source assembly 81.

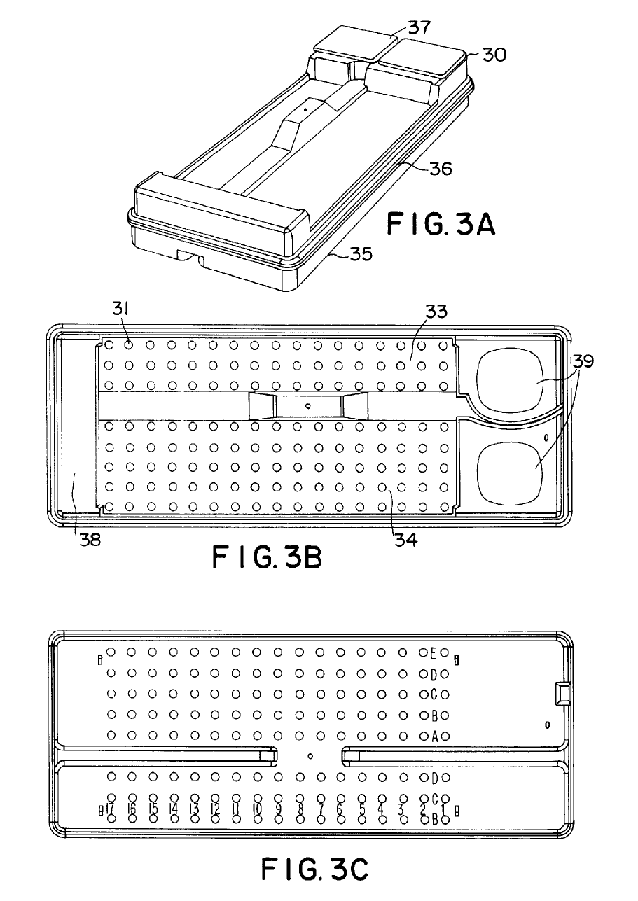

In FIGS. 3A-3C, the ID/AST panels 30 are supplied in a combination format. Each ID/AST combination panel 30 has reagent well positions capable of performing ID and AST testing on the same panel. The ID/AST panels 30 include the wells 31 which are segregated into an ID section 33 and an AST section 34. The ID section 33 of the ID/AST panel 30 consists of fifty-one wells 31. The AST section 34 of the ID/AST panel 30 consists of eighty-five wells 31 which contain dried antibiotics therein. However it will be understood by one of ordinary skill in the art that a separate ID panel or AST panel may be used instead of the ID/AST combination panel

In one embodiment, the test panel has at least one or more wells 31 contain a dried lincosamide agent therein in a concentration range on rehydration of 0.05 micrograms/ml to 8.0 micrograms/ml and is free of a macrolide agent, for example three wells contain dried clindamycin at a concentration on rehydration of 0.125 micrograms/ml. In addition, at least one or more wells 31 contain a dried macrolide agent therein in a concentration range on rehydration of 0.05 micrograms/ml to 8.0 micrograms/ml and is free of a lincosamide agent, for example two wells contain dried erythromycin at a concentration on rehydration of 0.4 micrograms/ml. At least one or more wells contain both a dried lincosamide agent and a dried macrolide agent therein in a concentration range on rehydration of 0.05 micrograms/ml to 8.0 micrograms/ml, for example one well contains dried clindamycin at a concentration on rehydration of 0.125 micrograms/ml and dried erythromycin at a concentration on rehydration of 0.4 micrograms/ml. In this particular embodiment, the wells containing the individual lincosamide agent, the individual macrolide agent and the wells containing the combination of both agents are located in the AST section of the test panel, however these wells can be located in either of the AST or ID section. In addition the same type of lincosamide agent (clindamycin) and the same type of macrolide agent (erythromycin) are used in the individual agent wells and in the wells containing the combination of both agents in this embodiment. However the individual agent wells may contain a different type of lincosamide agent and/or macrolide agent other than the type of lincosamide agent and the type of macrolide agent used in the wells containing the combination of both agents.

In another embodiment, where the microorganism to be tested is known to have a resistant MIC to erythromycin and susceptible MIC to clindamycin, the test panel has at least one or more wells contain a dried lincosamide agent and a dried macrolide agent therein in a concentration on rehydration range of 0.05 micrograms/ml to 8.0 micrograms/ml, for example one well contains both dried clindamycin at a concentration on rehydration of 0.125 micrograms/ml and dried erythromycin at a concentration on rehydration of 0.4 micrograms/ml.

The ID/AST panel 30 also includes a base 35, a chassis 36, a lid 37, and a cellulose acetate pad 38. Each ID/AST panel 30 also includes a panel label (not shown) which includes information to identify the complete manufacturing history of the particular ID/AST panel 30.

A barcode label provides information related to the ID/AST panel type and also has a unique sequence number for identification purposes. The barcode label can be provided in Code 128, numeric format or any other suitable barcode format.

Each ID/AST panel 30 is inoculated with a broth-suspended microorganism before being placed into the instrument 20. In practice, the microorganism is a processed and resuspended dilution of microbiological growth from primary culture in either an ID inoculum fluid or an AST inoculum fluid which is then poured into the test panel. The ID/AST panels 30 are inclined with the inoculation ports 39 at the top for filling. Separate inocula are added manually to the ID and AST ports 39. Each well 31 in the ID section 33 is inoculated with the ID inoculum fluid as the inoculum flows down the panel toward the pad 38. Each well 31 in the AST section 34 is inoculated with the AST inoculum fluid. The inocula flow down the ID/AST panel 30 in a serpentine fashion, filling the wells 31 as the liquid front progresses toward the pad 38. Each well 31 is vented, permitting liquid to fill the well 31. Each well 31 has a sharp, circular rim to separate a consistent quantity of liquid from the excess and to isolate each well 31 from liquid in adjacent wells 31. The pad 38 absorbs excess liquid.

The ID/AST panels 30 are inoculated with the inoculum fluids at a panel inoculation station (not shown). Each station holds two tubes of inoculum fluid (i.e., the ID inoculum fluid and the AST inoculum fluid) and supports one ID/AST panel 30. Gravity drives the inoculum fluids through the ID/AST panels 30.

The ID inoculum fluid and AST inoculum fluid comprise the reagent subsystem which includes all reagents required to process isolated bacterial colonies into prepared inocula for addition to the ID section 33 and the AST section 34 of the ID/AST panels 30.

The ID inoculum fluid is used for microorganism identification. A variety of ID inoculum fluids can be used, although a saline solution is preferred. A detergent may be added to enhance ID/AST panel 30 filling in the panel inoculation station. Preferably, the inoculum density for ID panel inoculation is at least 1×10⁵ cfu/ml. A variety of identification reagents may be used which include Phenol Red and Iodo-Nitro-Tetrazolium (INT). A variety of substrates may also be used which include 4-Methyl Umbelliferrone (4-MU) derivatives, Methyl-Amino-Coumarin (4-AMC) derivatives, para-Nitrophenol derivatives, and Esculin.

The AST inoculum fluid used for AST determination is a modified formulation of Mueller-Hinton broth. Preferably, the inoculum density for AST panel inoculation is at least 1×10⁵ cfu/ml. Different inoculum densities may be used for other embodiments of the present invention such as “rapid” AST test results. These are AST test results obtained within sixteen hours of ID/AST panel 30 inoculation.

A variety of AST indicators may be used. The preferred indicator for AST determinations in the present invention is alamarBlue™, a redox-buffered oxidation-reduction indicator. The indicator is added to the AST inoculum fluid and mixed just prior to addition of the microorganism sample to be tested by the instrument 20.

Control processor 70 interprets the data from the wells 31 for the purpose of detection, identification and susceptibility testing. The control processor uses three variable threshold levels to interpret this data: an absolute, a dynamic and relative threshold. When using the absolute threshold, a positivity assessment made by determining if the normalized well 31 reading is above (positive) or below (negative) a given predetermined value. When using the dynamic threshold, a reagent reaction determination is calculated using first- and second-differences or other mathematical manipulations of detection data related to the rate-of-change of signal increase as a function of time by determining when certain parameters of the calculated first- and/or second-differences have been exceeded. When using the relative threshold, a reagent reaction determination is made by setting a threshold a predetermined percentage above the starting signal level of the well 31 in question.

In operation, the ID/AST panels 30 are mounted and incubated in the carousel 50 of the instrument 20. As the visible light source assembly 80 and the UV light source assembly 81 are energized sequentially, one reading is taken corresponding to the red, green, blue and fluorescent wavelengths of light. Based on the rotation speed of the carousel 50, light intensity readings are taken at predetermined intervals by the optical measurement system 100.

For example, when the carousel 50 is driven by the drive system 56 at an angular velocity of 2.0 revolutions per minute (RPM), one rotation of the carousel 50 requires 30 seconds. Thus, to accumulate data for red, green, blue and UV wavelengths, two minutes are required. Accordingly, in this example, a complete set of data can be taken by the present invention every two minutes, Since it is possible to vary the angular velocity, different angular velocity may be used for different tests. For example, it may be desirable to accumulate UV data at 1.0 RPM (while other test data is accumulated at 2.0 RPM). In this case, a complete data set would require two and a half minutes to complete.

AST end-point results based on the well 31 readings can be obtained after a predetermined period of incubation, although the preferred end-point is after 18-24 hours of incubation.

According to an embodiment a positive result for the method to determine inducible resistance to macrolide-lincosamide-streptogramin b (iMLSb) is an microorganism which is resistant to erythromycin thus grows in the wells containing erythromycin at a concentration of no less than 8 μg/ml, susceptible to clindamycin thus grows in the wells containing clindamycin at a concentration of less than or equal to 0.5 μg/ml, and is resistant to the combination of clindamycin with erythromycin hence grows in wells containing both clindamycin with erythromycin.

According to another embodiment a positive result for the method to determine inducible resistance to macrolide-lincosamide-streptogramin b (iMLSb) in an microorganism known to have resistance to erythromycin, and be susceptible to clindamycin, is the presence of microorganism growth in the well containing both clindamycin with erythromycin.

The control processor 70 includes an ID taxa database that includes greater than 126 species for gram-negative organisms, and 103 species for gram-positive organisms. The control processor 70 also includes an AST taxa database equivalent to the ID taxa database for both gram-positive and -negatives. For the purposes of AST testing, the system also includes a database with all human and veterinary antimicrobics currently known. A plurality of optical filters are disposed between the test panel 30 and the light detection unit 100. The filters and pass only light emitted from, or absorbed by, the wells (shown as element 31 in FIG. 3B) having a predetermined bandwidth about a predetermined wavelength.

### EXAMPLE

A diverse collection of Staphylococci were tested according to an embodiment of the invention (Phoenix iMLSb) and using the “D-test”. The results of each test method for each microorganism as shown in Table 1 were then compared.

According to an embodiment of the invention, four wells in the AST section of a test panel contained dried clindamycin and erythromycin at the following respective concentrations upon rehydration (in μg/ml) 0.125 and 0.4; 0.125 and 0.8; 0.25 and 0.4; 0.25 and 0.80.

The AST inoculum fluid used for this AST determination is a modified formulation of Mueller-Hinton broth while alamarBlue™ was used as the indicator.

The inoculated ID/AST panel 30 was loaded into the carousel compartment and incubated at a temperature of 35° C. until a positive or negative result for the presence of inducible resistance to macrolide-lincosamide-streptogramin b (iMLSb) was obtained.

The D-test was carried out according to Clinical and Laboratory Standard Institute (CLSI) M100-S15, p. 114: Inducible clindamycin resistance can be detected using a disk approximation test by placing a 2-micro-gram clindamycin disk 15 mm away from the edge of a 15 micro-gram erythromycin disk on a standard blood agar plate used for the inoculum purity check. Following incubation, microorganisms that do not show flattening of the clindamycin zone would be reported as clindamycin susceptible. Microorganisms that show flattening of the clindamycin zone adjacent to the erythromycin disk (referred to as a “D” zone) have inducible clindamycin resistance. Such isolates should be reported as clindamycin resistant.

TABLE 1

Phoenix iMLSb

| | | Negative | Positive |
|---|---|---|---|
| D-Test | Negative | 56 | 1 |
| | Positive | 2 | 135 |

The results show close agreement between the embodiment of the invention and the “D-test” and as such validate the use of the embodiment as an alternative method for the detection of inducible resistance to macrolide-lincosamide-streptogramin b (iMLSb) within a microorganism.

While the present invention has been described above in terms of specific embodiments, it is to be understood that the invention is not intended to be confined or limited to the embodiments disclosed herein. On the contrary, the present invention is intended to cover various methods, structures and modifications thereof included within the spirit and scope of the appended claims.

Although only a few exemplary embodiments of the present invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

What is claimed is:

1. A test panel comprising
a plurality of wells,
a lincosamide agent, and
a macrolide agent,
wherein one or more of said wells contains both said lincosamide agent and said macrolide agent.

2. A test panel as in claim 1 wherein one or more of said wells comprises said lincosamide agent and is free of said macrolide agent.

3. A test panel as in claim 2 wherein at least three of said wells contain said lincosamide agent and are free of said macrolide agent.

4. A test panel as in claim 1 wherein said lincosamide agent is clindamycin.

5. A test panel as in claim 4 wherein said clindamycin is in a dehydrated state.

6. A test panel as in claim 5 wherein the concentration on rehydration of said clindamycin is within the range of 0.05 micrograms/ml to 8 micrograms/ml.

7. A test panel as in claim 5 wherein the concentration on rehydration of said clindamycin is 0.125 micrograms/ml.

8. A test panel as in claim 1 wherein one or more of said wells comprises said macrolide agent and is free of said lincosamide agent.

9. A test panel as in claim 8 wherein at least three of said wells contain said macrolide agent and are free of said lincosamide agent.

10. A test panel as in claim 1 wherein said macrolide agent is erythromycin.

11. A test panel as in claim 10 wherein said erythromycin is in a dehydrated state.

12. A test panel as in claim 11 wherein the concentration on rehydration of said erythromycin is within the range of 0.05 micrograms/ml to 8 micrograms/ml.

13. A test panel as in claim 11 wherein the concentration on rehydration of said erythromycin of said is 0.4 micrograms/ml.

14. A method comprising
inoculating a test panel having a plurality of wells with a test microorganism, wherein one or more of said wells contains both a lincosamide agent and a macrolide agent and one or more of said wells contains a lincosamide agent and is free of a macrolide agent, and one or more of said wells contains a macrolide agent and is free of a lincosamide agent,
placing said test panel into a testing system
incubating said test panel at an appropriate temperature, and
monitoring the growth rate of said test microorganism in said wells,
comparing the growth rate of said test microorganism in said wells containing said lincosamide agent and free of a macrolide agent, and the growth rate of said test microorganism in said wells containing said macrolide agent and free of a lincosamide agent, and the growth rate of said test microorganism in said wells containing both said lincosamide agent and said macrolide agent,
such that the presence of resistance to macrolide-lincosamide-streptogramin b (iMLSb) in said test microorganism is indicated by resistance in said wells containing said macrolide agent and free of a lincosamide agent, susceptibility in said wells containing said lincosamide agent and free of a macrolide agent, and resistance in said wells containing both said lincosamide agent and said macrolide agent.

15. A method as in claim 14 wherein at least three of said wells contain said lincosamide agent and are free of a macrolide agent.

16. A method as in claim 14 wherein said lincosamide agent is clindamycin.

17. A method as in claim 14 wherein at least three of said wells contain said macrolide agent and are free of a lincosamide agent.

18. A method as in claim 14 wherein said macrolide agent is erythromycin.

19. A method as in claim 14 wherein said testing system is an automated, a semi-automated or manual testing system.

20. A method as in claim 19 wherein said automated testing system is a microorganism identification (ID) and antimicrobial susceptibility determination (AST) system.

21. A method as in claim 14 wherein said testing system is a microorganism identification (ID) and antimicrobial susceptibility determination (AST) system.

22. A method as in claim 14 wherein said testing system is an antimicrobial susceptibility determinations (AST) system.

23. A method comprising
inoculating a test panel having a plurality of wells with a test microorganism with resistance to erythromycin and susceptibility to clindamycin, wherein one or more of said wells contains both a lincosamide agent and a macrolide agent,
placing said test panel into a testing system
incubating said test panel at an appropriate temperature, and
monitoring the growth rate of said test microorganism in said wells, wherein the presence of growth within said one or more of said wells containing both said lincosamide agent and said macrolide agent, indicates the presence of resistance to macrolide-lincosamide-streptogramin b (iMLSb).

24. A method as in claim 23 wherein said testing system is an automated, a semi-automated or manual testing system.

25. A method as in claim 24 wherein said automated testing system is a microorganism identification (ID) and antimicrobial susceptibility determinations (AST) system.

26. A method as in claim 23 wherein said testing system is a microorganism identification (ID) and antimicrobial susceptibility determinations (AST) system.

27. A method as in claim 23 wherein said testing system is an antimicrobial susceptibility determinations (AST) system.

28. A test panel comprising
a plurality of wells,
at least one lincosamide agent, and
at least one macrolide agent,
wherein one or more of said wells contains both a lincosamide agent and a macrolide agent and one or more of said wells contains a lincosamide agent and is free of a macrolide agent, and one or more of said wells contains a macrolide agent and is free of a lincosamide agent.
